(19) United States
(12) Patent Application Publication
(10) Pub. No.: US 2023/0226335 A1
(43) Pub. Date: Jul. 20, 2023

(54) ACCESS DEVICE

(71) Applicant: ABIOMED, Inc., Danvers, MA (US)

(72) Inventors: Adam Finck, Danvers, MA (US); Emilia Jahangir, Danvers, MA (US); Charles Anthony DeLorenzo, Danvers, MA (US); Glen Fantuzzi, Danvers, MA (US); Brian Chouinard, Danvers, MA (US)

(73) Assignee: ABIOMED, Inc., Danvers, MA (US)

(21) Appl. No.: 18/093,878

(22) Filed: Jan. 6, 2023

Related U.S. Application Data
(60) Provisional application No. 63/297,506, filed on Jan. 7, 2022.
(60) Provisional application No. 63/313,791, filed on Feb. 25, 2022.
(60) Provisional application No. 63/344,300, filed on May 20, 2022.

(51) Int. Cl. A61M 39/06

(52) U.S. Cl. CPC A61M 39/06; A61M 2039/0297

(57) ABSTRACT

The present disclosure is drawn to an access device that includes a cannula with a joint lumen therethrough, and a hub coupled to a proximal end of the cannula. The hub includes at least two arms. The first arm may have a first lumen and a first hemostatic valve, the first lumen operably connected to the joint lumen, where the first lumen and the first hemostatic valve are configured for passage of a medical device, and the second arm may be coupled to the first arm and have a second lumen, the second lumen operable connected to the joint lumen, the second arm configured to be operably coupled to an external medical device, such as an extracorporeal membrane oxygenation (ECMO) device.

### CROSS-REFERENCE TO RELATED APPLICATIONS

The present application claims priority to US Provisional Patent Application Nos. 63/297,506, filed Jan. 7, 2022, 63/313,791, filed Feb. 25, 2022, and 63/344,300, filed May 20, 2022, the entirety of each of which are incorporated herein by reference in their entirety.

### TECHNICAL FIELD

The present disclosure is drawn to surgical access devices, and specifically to an access device capable of being used to facilitate the introduction of medical devices into a patient and to facilitate the circulation of blood through an extracorporeal device.

### BACKGROUND

Extracorporeal Membrane Oxygenation (ECMO) involves the use of a mechanical circulatory device for patients experiencing cardiogenic shock, or other forms of hemodynamic deterioration. Ventricular assist devices (VADs) and catheter-based devices (such as intravascular blood pumps) may be used to unload the heart (e.g., the left ventricle).

Access devices are commonly used in surgical procedures to facilitate the introduction of a medical instruments into the body's natural biological blood vessels, cavities, etc. These access devices include, for example, devices that facilitate the introduction of guide wires, balloon catheters, or other catheter-based medical devices into the vasculature of the human body. These access devices also can be used to facilitate the extracorporeal circulation of blood, such as when utilizing an extracorporeal membrane oxygenation (ECMO) device (including, e.g., veno-arterial ECMO (VA-ECMO) or veno-venous ECMO (VV-ECMO) devices).

### BRIEF SUMMARY

According to a first aspect of the present disclosure, an access device can be provided to allow two or more medical devices to be inserted through the device into a patient. The access device may include a hub that is configured to be coupled to a proximal end of a cannula having a proximal end and a distal end and a joint lumen therethrough. The access device may also include, where the hub may have a first arm and a second arm, the second arm coupled to the first arm. The first arm may have a first lumen operably connected to the joint lumen, and a first hemostatic valve configured for passage of a medical device. The second arm may be operably connected to the joint lumen and may be configured to be operably coupled to an extracorporeal membrane oxygenation (ECMO) device. In some embodiments, the cannula may be coupled to the hub via a threaded connection or a barbed connection. In some embodiments, the medical device may be a guide wire, a guide catheter, a balloon catheter, or a catheter-based heart pump.

In some embodiments, the cannula may be a joint cannula which may comprise a plurality of sections. In some embodiments, the joint cannula may comprise a first joint cannula section having a proximal end coupled to the hub and a second joint cannula section having a proximal end coupled to a distal end of the first joint cannula section. In some embodiments, the first joint cannula section, the second joint cannula section, or both, may be semi-rigid bodies.

In some embodiments, the second arm may be configured to be operably coupled to the ECMO device via an additional cannula. In some embodiments, the additional cannula may be a flexible cannula.

In some embodiments, a first angle formed between a central axis of the first arm and a central axis of the second arm may be less than 90 degrees. In some embodiments, a second angle formed between a central axis of the first lumen and a central axis of the second lumen may be less than 90 degrees. In some embodiments, the first angle, the second angle, or both may be between 30 degrees and 60 degrees.

In some embodiment, the first lumen and second lumen connect to form a junction.

In some embodiments, the access device may also comprise a clamp configured to allow a user to clamp off the second arm.

In some embodiments, the access device may also comprise a fixation feature, such as a butterfly pad or a suture ring. In some embodiments, the fixation feature may be configured to be axially stationary with respect to the cannula. In some embodiments, the fixation feature may be movably positioned along the cannula.

In some embodiments, the joint lumen may have an inner diameter ID, where 3 mm≤ID≤36 mm. In some embodiments, the joint lumen may have an inner diameter ID≤6.5 mm. In some embodiments, the joint lumen may have an inner diameter ID, where 5 mm≤ID≤6.5 mm.

In some embodiments, the cannula may have a wall thickness of between 0.2 mm and 0.4 mm. In some embodiments, some or all of the cannula may be reinforced with coiled wire, braided wire, or a precision-cut hypotube. In some embodiments, the cannula may comprise a low-friction polymer coating (such as Polytetrafluoroethylene (PTFE)) on an inner surface of the joint lumen. In some embodiments, the cannula may comprise a thermoplastic polyurethane, a nylon, or a polyamide block polymer. In some embodiments, the cannula may comprise a radiopaque material.

In some embodiments, the cannula may include a straight cannula. As will be appreciated, in other embodiments, at least a portion of the cannula may be bent and/or curved. In some embodiments, the bend and/or curve may be disposed a prescribed distance along the catheter length.

In some embodiments, the cannula may have one or more lumens in a distal portion of the cannula, where each of the one or more lumens may extend from an outer surface of the cannula to the joint lumen through a sidewall of the cannula.

In some embodiments, the cannula may be configured to receive a dilator assembly. In some embodiments, the dilator assembly can be passed through the first or second arms (e.g., through the arm offering ECMO support or for passage of a medical device. In some embodiments, the hub may comprise a third lumen operably connected to the first lumen, the second lumen, or both. In some embodiments, this third lumen may be configured to allow a fluid to enter or exit the cannula through the hub. In some embodiments, this third lumen may be configured to connect to an external accessory, such as a distal leg perfusion cannula, a pressure bag, or an infusion pump. In some embodiments, this third lumen may be connected to a valve. The valve may be between the hub and an external accessory.

According to a second aspect of the present disclosure, a method for using the above-described access devices is provided. The method may comprise providing any embodiment of an access device according to the first aspect of the present disclosure, inserting a medical device into the first arm of the access device and into a patient, and then oxygenating blood with an extracorporeal membrane oxygenation (ECMO) device coupled to the second arm of the access device, where the blood flows through the joint lumen and the second lumen of the access device. In some embodiment, the insertion step may include inserting the medical device through the first hemostatic valve, the first lumen, and the joint lumen. In some embodiments, the medical device may be an intravascular blood pump.

According to a third aspect of the present disclosure, a kit may be provided. The kit may comprise or consist of any embodiment of an access device according to the first aspect of the present disclosure, an extracorporeal membrane oxygenation (ECMO) device configured to be coupled to the second arm of the single access device, and at least one medical device configured to be inserted through the first hemostatic valve, the first lumen, and the joint lumen of the access device. In some embodiments, the medical device may be an intravascular pump. In some embodiments, the kit also may include a needle to enable the physician to gain access to the artery or vein. In some embodiments, the kit also may include a guidewire to enable placement of the cannula into the vasculature. The kit also may include one or more dilators at subsequent sizes to sequentially expand the vascular prior to insertion of the described device.

### BRIEF DESCRIPTION OF DRAWINGS

FIG. 1A is a cutaway views of an embodiment of an access device connected to an ECMO device.

FIG. 1B is a cross-sectional view of a cannula according to one embodiment.

FIG. 1C is a cutaway view of an embodiment of an access device according to some embodiments.

FIGS. 2A-2C are cutaway views of embodiments of cannulas of an access device.

FIG. 3 is a cutaway view of an embodiment of a joint cannula comprising two or more sections.

FIG. 4A is a cutaway view of an embodiment of a hub.

FIGS. 4B and 4C are cutaway views of embodiments of different connection options for a hub.

FIGS. 4D and 4E are cutaway views of embodiments of an access device according to another embodiment.

FIG. 4F is a cutaway view of an embodiment of a hub.

FIG. 4G is a cutaway view of an embodiment of a hub connected to an ECMO device.

FIGS. 5A and 5B are schematic representations of embodiments of different lumen configurations internal to a hub.

FIG. 6 is a flowchart of an embodiment of a method.

FIG. 7 is a schematic of an embodiment of an access device inserted into a patient.

FIG. 8 is a cutaway view of a modular access device according to some embodiments.

FIGS. 9A-9C illustrate embodiments of a modular access device.

FIG. 10 illustrates an access device according to one embodiment.

FIG. 11 illustrates an embodiment of a modular access system according to one embodiment.

FIG. 12A illustrates an access device having an inflatable valve.

FIGS. 12B and 12C illustrate different configurations of an inflatable valve.

### DETAILED DESCRIPTION

Cardiogenic shock is the leading cause of death for patients with acute myocardial infarction (AMI) who reach the hospital alive. Cardiogenic shock is caused by a heart malfunction or problem, which leads to an inability of the heart to eject enough blood for the body. In some instances, ventricular assist devices (VADS) and catheter-based VADS (such as intravascular blood pumps) may be used to mechanically unload the heart (e.g., the left ventricle).

Extracorporeal Membrane Oxygenation (ECMO) allows for gas exchange of the blood when the lungs do not work properly and may involve the use of a mechanical circulatory device for patients experiencing oxygenation issues. In some instances, ECMO may be used for patients experiencing oxygenation issues due to cardiogenic shock, or from other forms of hemodynamic deterioration. In some instances, use of such devices may result in an increase in left ventricular afterload.

As described herein, in some instances, patients may need both ECMO support and a VAD. In some instances, such support may take place at the same time, although in some instances, a patient may require ECMO support prior to VAD support. Traditionally, this requires multiple insertion points, which may add additional time, complexity, and/or risks to a surgical procedure. As such, the inventors have recognized the benefit of an access device capable of being used to facilitate the introduction of medical devices and/or to facilitate the circulation of blood through an extracorporeal device is useful and desirable.

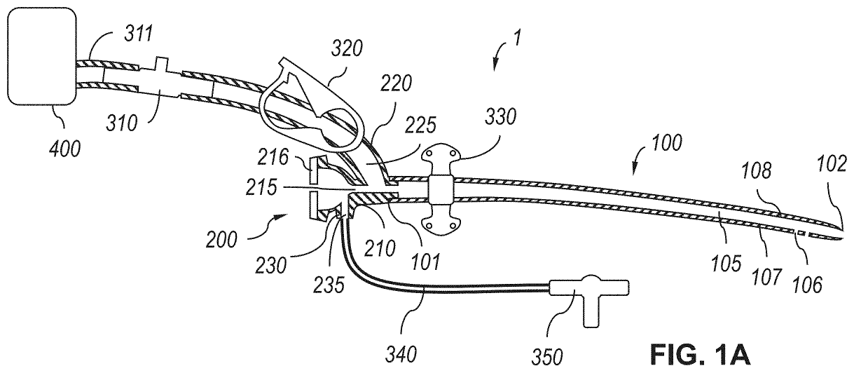

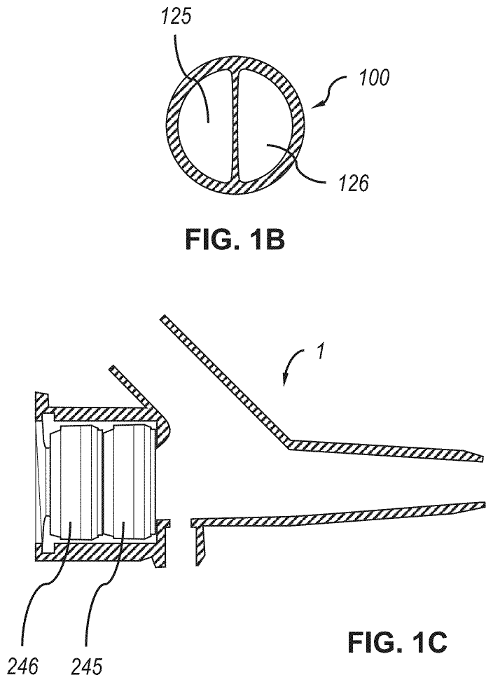

Referring to FIG. 1A, an access device 1 according to embodiments of the present disclosure is shown. As shown in this view, in some embodiments, the access device includes a first arm 210 and a second arm 220 configured to allow one or more medical devices to be inserted through the device and into a patient. Various medical devices may be utilized. For example, in some embodiments, catheter-based medical devices may be inserted into the patient. Further, in some embodiments, one arm may be used for inserting one or more medical devices, while the other arm may allow for, e.g., ECMO support. As will be appreciated, in some embodiments, the access device may allow a plurality of medical devices to be inserted through the device into a patient. As described herein, the access device may allow for simultaneous ECMO support and insertion of medical device and tandem ECMO support and insertion of a medical device (e.g., insertion before and/or after completion of ECMO support). The device also may allow for a medical device (e.g., a VAD) to remain installed in a patient and through the access device while ECMO support is discontinued.

In some embodiments, the access device may be connected to a shared cannula 100 having a proximal end 101 and a distal end 102. As will be appreciated, the cannula may be permanently attached to the access device in some embodiments or may be attachable to the access device in other embodiment (e.g., by a clinician). In embodiments in which the cannula is attachable to the access device, the cannula may be configured to be fixedly attached to the access device.

In some embodiments, the shared cannula may include a joint lumen 105 (sometimes referred to as a shared lumen) therethrough. For purposes herein, the joint lumen may include a single lumen extending along the length of the shared cannula that may be used to both pass one or more medical devices and to pass blood therethrough (e.g., from an ECMO circuit). In other embodiments, the joint lumen may include more than one lumen extending along the length of the shared cannula. For example, in some embodiments, the cannula may include two parallel lumens extending along the length of the shared cannula (see, e.g., the cross-section of an embodiment of a shared cannula in FIG. 1B, showing two lumens through at least part of the shared cannula). In such embodiments, the medical devices may extend through a first lumen and the ECMO circuit may be connected to the second lumen. In another embodiment, the shared cannula may include a first portion with a single lumen and a second portion with more than one lumen (e.g., two parallel lumens). In such embodiments, the single lumen may communicate with each of the parallel lumens.

Although shown and described as being attached to a shared cannula, it will be appreciated that the access device may be attached to the patient via other suitable manners. For example, in some embodiments, the access device may be connected to a graft, which is thereafter attached to the patient.

As also shown in FIG. 1A, the access device may include a hub 200 that is configured to be coupled to a proximal end 101 of the cannula. As shown in this view, the hub includes the first arm 210 and the second arm 220, the second arm being coupled to the first arm.

The first arm 210 may have a first lumen 215 operably connected to the cannula (e.g., to the joint lumen 105). A proximal end of the first arm may include a first hemostatic valve 216 configured for passage of a medical device. In some embodiments the hemostatic valve may be configured to minimize and/or prevent blood leakage via the first arm. In some embodiments, the medical device to be inserted may be a guide wire, a balloon catheter, or a catheter-based heart pump. In some embodiments, the medical device to be inserted may be an intravascular heart pump. As will be appreciated, other catheter-based medical devices also may be insertable via the first arm. As will be further appreciated, in embodiments in which the cannula includes more than one lumen extending therethrough, the first lumen 215 may be connected to a corresponding first lumen of the cannula (see, e.g., first lumen 125 of FIG. 1B).

In some embodiments, a portion of each of a plurality of medical devices may be present within the first lumen and joint lumen of the access device at the same time. In some embodiments, the access device (e.g., first arm) may include one or more features for holding the position of the medical device relative to the access device when the medical device is in the patient. For example, the access device may include one or more locking features to lock the placement of the medical device when the medical device is in the patient. As will be appreciated, the locking feature may be configured to be disengaged to allow removal of the medical device.

Although the first arm is shown as having only a single hemostatic valve, in other embodiments, the first arm may include more than one hemostatic valve. For example, as shown in FIG. 1C, the first arm may include first hemostatic valve 245 and second hemostatic valve 246. In such embodiments, the second hemostatic valve 246 may provide redundancy in minimizing and/or preventing bleeding out of the first arm (e.g., when one or more medical devices are inserted through the first arm.

In some embodiments, the hemostatic valves may both be oriented in the same direction, although the valves may be oriented in different directions. In embodiments having only a single hemostatic valve, the valve may be oriented as that shown in FIG. 1A, or may be flipped and oriented in the opposite direction.

The second arm 220 may be operably connected to the joint lumen 105 and may be configured to be operably coupled (e.g., through one or more additional connectors 310, cannulas 311, etc.) to an external device 400 (such as an extracorporeal membrane oxygenation (ECMO) device). As with the above, in embodiments in which the cannula includes more than one lumen extending therethrough the lumen 205 of the second arm may be connected to the second lumen (see, e.g., second lumen 126 in FIG. 1B) of the cannula.

In some embodiments, the access device 1 may also include a clamp 320 configured to allow a user to clamp off the second arm. In some embodiments, the clamp 320 may be integral to the second arm. In some embodiments, the clamp 320 may be removably attached to the second arm. In some embodiments, the clamp may include a Roberts clamp, although other suitable clamps may be used in other embodiments. It will be appreciated that the second arm may include other arrangements for controlling blood flow through the second arm. For example, in some embodiments, the access device may include built-in valving (e.g., a stop cock) or clamping to control flow.

In some embodiments, the access device may also comprise a fixation feature 330, such as a butterfly pad, a suture pad, or a suture ring. In some embodiments, the fixation feature may be configured to be axially stationary with respect to the cannula. In some embodiments, the fixation feature may be rotatable about the cannula. In some embodiments, the fixation feature may be movably positioned along the shared cannula 100.

In some embodiments, the shared cannula 100 may have one or more side openings 106 in a distal portion of the cannula (e.g., closer to the distal end 102 of the cannula than the proximal end 101), where each of the one or more side openings 106 may extend from an outer surface 107 of the cannula to the joint lumen 105 through a sidewall 108 of the cannula. In some embodiments, such side openings may provide an alternative path for blood flow through the cannula, such if the joint lumen were to become restricted (e.g., the cannula were pressed up against a vascular wall). In some embodiments, the side holes may be between 2 and 4 mm, such as 3 mm in size. In some embodiments, there may be 2, 4, 6, 8, 10, 12, or more side openings along the length of the tip. For example, in an illustrative embodiment, there may be 6 openings along the distal portion of the cannula.

In some embodiments, the distal portion of the cannula may be reinforced. In some embodiments, this may provide stability to the distal portion of the tip (e.g., to reduce the possibility of a change in diameter of the cannula and/or to assist with insertion of a medical device).

In some embodiments, the cannula may have a single stiffness, while in other embodiments, a stiffness of the cannula may be varied along a length of the cannula. For example, in some embodiments, the cannula may have a stiffer section near the proximal end and a softer section near the distal end.

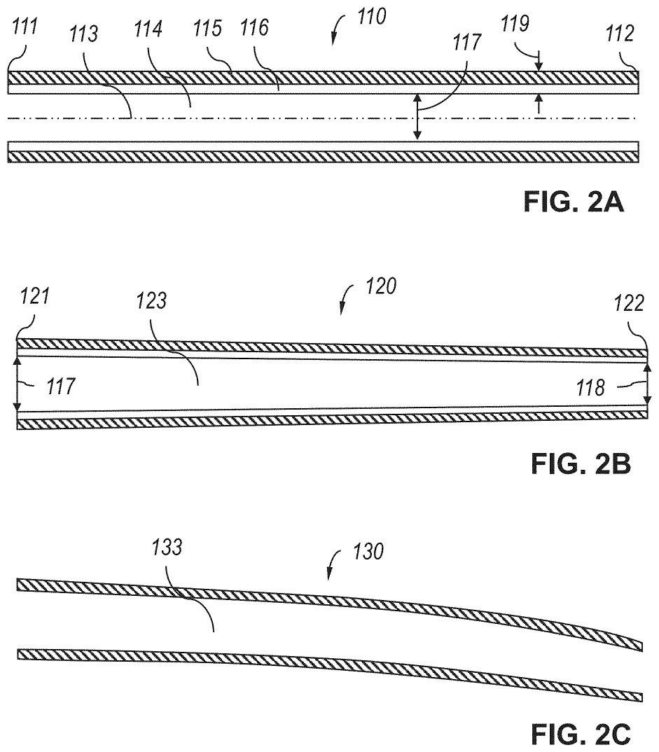

Referring to FIGS. 2A-2C, it can be seen that the shared cannula 100 may have different configurations. For example, in some embodiments, the cannula may comprise or consist of a straight cannula 110, 120. In some embodiments, the straight cannula 110 defines a joint lumen 114 that has an inner diameter 117 that is substantially constant at all points along the joint lumen's central axis 113 from the proximal end 111 to the distal end 112. In other embodiments, the straight cannula 120 does not have a substantially constant inner diameter. For example, as seen in FIG. 2B, the straight cannula 120 may be somewhat trapezoidal, having straight sidewalls but defining a joint lumen 123 that has an inner diameter 117 at a proximal end 121 that is larger than an inner diameter 118 at a distal end 122.

In some embodiments, the cannula may comprise or consist of a curved cannula 130 (see FIG. 2C), where the curved cannula forms a joint lumen 133 that may be formed to have at least one predefined radius of curvature. In some embodiments the cannula may have a bent portion. As will be appreciated, the cannula may have other suitable arrangements in other embodiments.

In some embodiments, the joint lumen 105, 114, 123, 133 may have an average inner diameter ID, where 3 mm≤ID≤36 mm. In some embodiments, the joint lumen may have an average inner diameter ID≤6.5 mm. In some embodiments, the joint lumen may have an average inner diameter ID, where 5 mm≤ID≤6.5 mm. In some embodiments, cannulas may be formed having different outer diameters (e.g., 16.5 Fr, 17.5 Fr, 19 Fr, and/or 21 Fr).

In some embodiments, the cannula may have a wall thickness 119 of between 0.2 mm and 0.4 mm. In some embodiments, the wall thickness may be substantially constant. In some embodiments, the wall thickness in one portion of the cannula may be thicker than the wall thickness in a different portion of the cannula (excepting any rounded or thinned ends of the cannula).

In some embodiments, the cannula may include one or more layers. In some embodiments, the cannula may comprise an inner layer 116 and an outer layer 115 (sometimes referred to as a “jacket”). In some embodiments, some or all of the cannula may be reinforced with coiled wire, braided wire, or a precision-cut hypotube. In some embodiments, the outer layer 115 may include coiled wire, braided wire, or a precision-cut hypotube. In such embodiments, the cannula may be reinforced (e.g., via nitinol or stainless steel). In some embodiments, the cannula may include a low-friction polymer coating (such as Polytetrafluoroethylene (PTFE) or HDPE) on an inner surface of the joint lumen. In some embodiments, the inner layer 116 may include a low-friction polymer coating (such as Polytetrafluoroethylene (PTFE)). In some embodiments, one or more of the layers forming the cannula may include a thermoplastic polyurethane, a nylon, or a polyamide block polymer. In some embodiments, the outer layer may include a hydrophilic coating.

In some embodiments, the cannula may include a tapered extension. In some embodiments, the extension may be heat molded over a reinforced body. In some embodiments, this may allow for modular connection of the cannula, such as to a barbed connector, as described herein.

In some embodiments, the cannula may comprise a radiopaque material. In some embodiments, the radiopaque material is a metallic element. In some embodiments, the radiopaque material is tungsten, silver, tantalum, or tin. In some embodiments, the radiopaque material is a tungsten powder. In some embodiments, the radiopaque material may be combined with a polymer (such as a polyurethane). In some embodiments, the radiopaque material is arranged in bands offset axially from each other along some or all of the length of the cannula.

In some embodiments, the cannula may be configured to receive a dilator assembly.

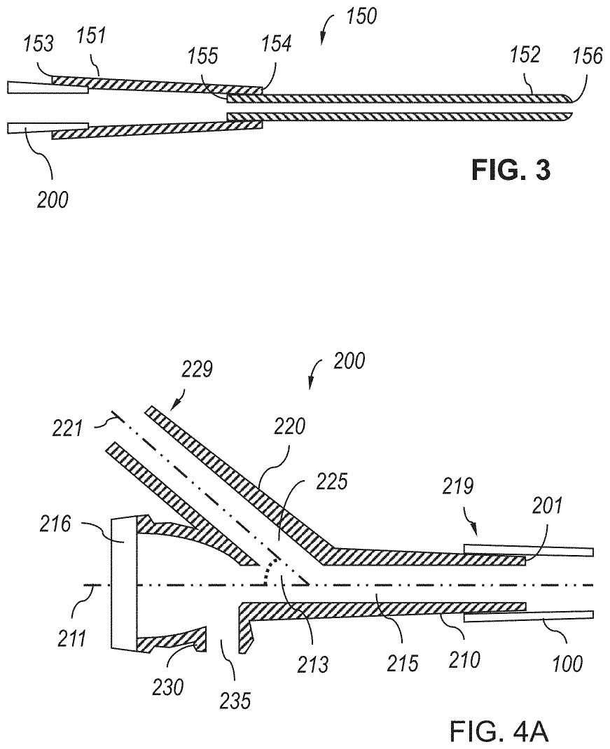

As seen in FIG. 3, in some embodiments, the cannula 150 which may include a plurality of sections. In some embodiments, the cannula 150 may comprise a first cannula section 151 having a proximal end 153 coupled to the hub 200 and a second cannula section 152 having a proximal end 155 coupled to a distal end 154 of the first cannula section 151. In some embodiments, the distal end 156 of the second cannula section 152 may not be coupled to any other joint cannula section. In some embodiments, the distal end 156 of the second cannula section 152 may be coupled to an additional cannula section (not shown). In some embodiments, the first cannula section 151, the second cannula section 152, or both, may be semi-rigid bodies. As will be appreciated, although shown as having two connected portions, it will be appreciated that the cannula may include more than two connected portions in some embodiments. As will be further appreciated, the cannula sections may be fixedly joined together before the cannula is used by a clinician. In other embodiments, the first and second cannula portions may be part of the kit and may be attached together by a clinician.

In some embodiments, the first cannula section is configured to have a stiffness than is greater than the stiffness of the second cannula section.

In some embodiments, the first cannula section is configured to be a straight cannula. In some embodiments, the second cannula section is straight cannula. In some embodiments, the second cannula section is a curved cannula.

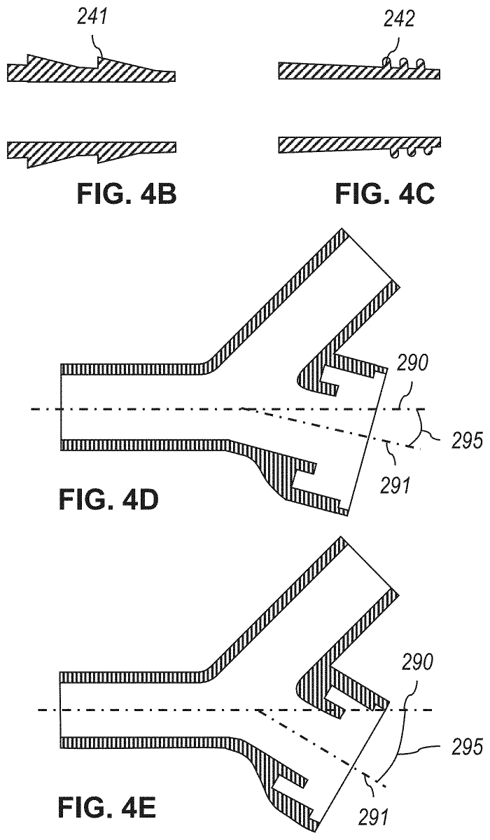

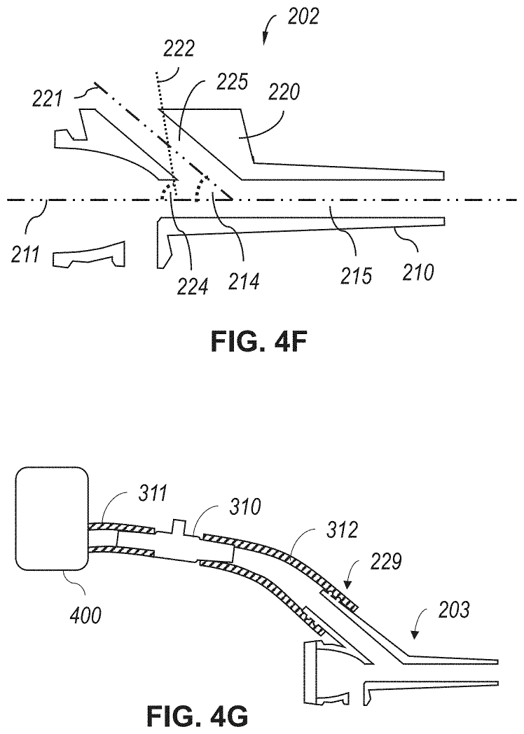

Referring to FIGS. 4A-4C, the shared cannula 100 may be coupled to a distal end 201 of the hub 200. In some embodiments, as shown in FIG. 4A, the distal portion 219 of the first arm 210 of the hub may be configured to be coupled to the cannula. In other embodiments, the proximal portion 229 of the second arm 220 of the hub may be configured to be coupled to a cannula. As seen in FIG. 4A, the distal portion 219 of the first arm or the proximal portion 229 of the second arm may be substantially smooth. As seen in FIG. 4B, these portions (e.g., distal portion 219 and proximal portion 229) may have one or more barbs 241, to allow a cannula to be coupled to the hub via a barbed connection. As seen in FIG. 4C, these portions (distal portion 219 and proximal portion 229) may have one or more threads 242, to allow a cannula to be coupled to the hub via a threaded connection.

Referring again to FIG. 4A, in some embodiments, the first arm and first lumen may be coaxial, and the second arm and second lumen may be coaxial. In such embodiments, an angle 213 at the proximal end formed between (a) a central axis 211 of a distal portion of the first lumen 215 and (b) a central axis 221 of second lumen 225 may be less than 90 degrees. In some embodiments, this angle 213 may be 15-30 degrees. In some embodiments, this angle 213 may be 30 degrees.

In some embodiments, the first lumen is straight. In some embodiments, the first lumen bends, such as at the junction of the first lumen and the second lumen. In some embodiments, an angle 295 formed between (a) a central axis 290 of a distal portion of the first lumen and (b) a central axis 291 of a proximal portion of the first lumen is greater than 0 and less than 90 degrees. For example, the angle 295 may be 15 degrees in some embodiments (see FIG. 4D) while in other embodiments the angle 295 may be 30 degrees (see FIG. 4E).

Referring to FIG. 4F, in some embodiments, the hub 202 may be configured such that the first arm and first lumen may not be coaxial, and/or the second arm and second lumen may not be coaxial. In FIG. 4F, a non-limiting embodiment is shown where the hub 202 is configured so the first arm and first lumen are coaxial, but the second arm and second lumen are not. In some embodiments, a first angle 224 formed between a central axis 211 of the first arm and a central axis 222 of the second arm 220 may be less than 90 degrees. In some embodiments, a second angle 214 formed between a central axis 211 of the first lumen 215 and a central axis 221 of the second lumen 225 may be less than 90 degrees. In some embodiments, the first angle 224, the second angle 214, or both may be between 30 degrees and 60 degrees.

As seen in FIGS. 4A and 4G, in some embodiments, the second arm 220 of the hub 203 may be configured to be operably coupled to an external device 400 (e.g., an ECMO device) via one or more additional cannulas 311, 312 and one or more connectors 310. As seen in FIG. 4G, in some embodiments, the proximal end of the second arm 220 may be connected to a distal end of an additional cannula 312. The proximal end of the additional cannula 312 may be connected to a connector 310, which may be connected to one or more additional cannulas 311 before connecting with the external device 400 (e.g., an ECMO device).

In some embodiments, each additional cannula 311, 312 may independently be a flexible cannula. In some embodiments, all additional cannulas 311, 312 may be flexible cannulas.

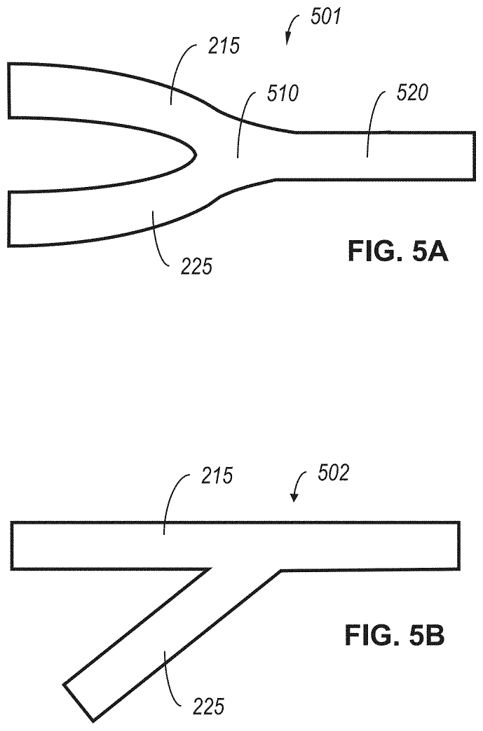

Referring to FIGS. 5A and 5B, the first lumen and second lumen may be connected in a variety of configurations 501, 502. In some non-limiting embodiments, the lumens may have a configuration 501 such that the first lumen and the second lumen connect to form a junction 510, where the proximal portion of the first lumen 215 and the second lumen 225 extend proximally from the junction, and where a distal portion of the third lumen 520 (distal to the junction of the first lumen and the second lumen) extends distally towards the distal end of the hub. In such embodiments, a central axis of the distal portion of the third lumen (not shown) may not be coaxial with either the central axis of the proximal portion of the first lumen or the second lumen. In some non-limiting embodiments, the lumens may have a configuration 502 such that the second lumen 225 intersects with the first lumen 215 as the first lumen extends distally towards the distal end of the hub.

Referring again to FIG. 1A, in some embodiment, the hub 200 may comprise a third lumen 235 operably connected to the first lumen 215, the second lumen 225, or both. In some embodiments, the third lumen 235 may be configured to connect to an external accessory, such as a distal leg perfusion cannula, a pressure bag, or an infusion pump.

In some embodiments, the third lumen 235 may be configured to allow a fluid to enter or exit the shared cannula 100 through the hub 200. In some embodiments, the third lumen 235 may be coupled to tubing 340. In some embodiments, the third lumen 235 may be connected, directly or indirectly, to a valve 350. In some embodiments, a valve 350 may be disposed between the hub 200 and an external accessory. In some embodiments, the valve may be a three-way stopcock.

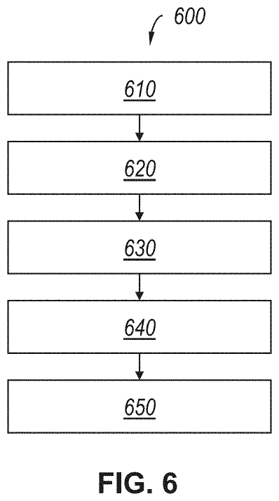

According to other embodiments, a method for using the above-described access devices is provided. Referring to FIG. 6, embodiments of the method 600 may first include providing 610 any embodiment of an access device as described herein. This access device may be surgically attached to a patient, where at least a portion of the cannula is inserted 620 into the patient through a single insertion site.

The method may then include an insertion step 630, where a medical device (such as an intravascular blood pump, etc.) may be inserted into the first arm of the access device and then into a patient through the cannula. In some embodiment, insertion step 630 may include inserting a medical device through the first hemostatic valve, the first lumen, and the joint lumen.

The method also may include coupling 640 an ECMO device to the second arm of the access device, after which the method includes oxygenating 650 blood with the ECMO device, where the blood flows through the joint lumen and the second lumen of the access device. As will be understood, insertion step 630 and coupling 640 may be completed in any order. In some embodiments, coupling 640 and oxygenating 650 may be completed before the insertion step 630 is completed.

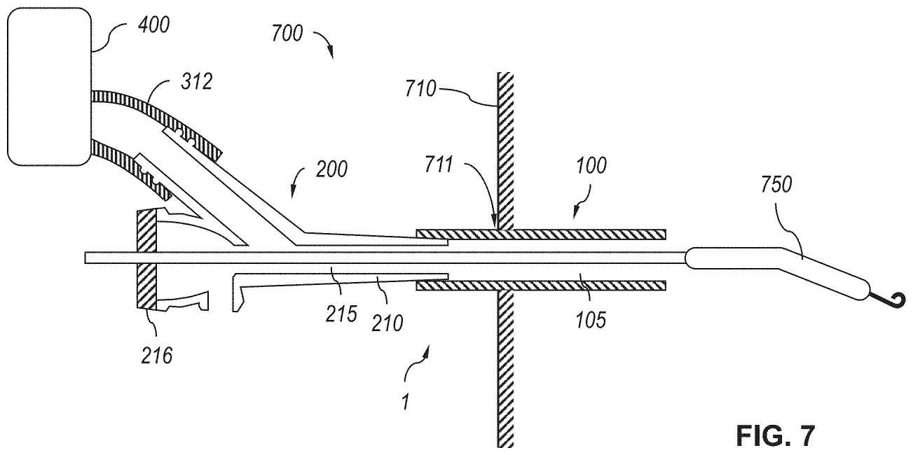

The disclosed method can be seen visually in FIG. 7, where an embodiment of a system 700 comprising the access device 1 can be seen inserted into a patient. There, at least a portion of the shared cannula 100, coupled to the hub 200, has been passed through the surface of a patient's skin 710 at an insertion site 711, and into the patient.

A medical device 750 (here, an intravascular blood pump) has been inserted into the first arm 210 of the access device 1 and then into a patient through the shared cannula 100. Specifically, the medical device 750 has been inserted through the first hemostatic valve 216, the first lumen 215, and the joint lumen 105.

In some embodiments, a portion of the cannula may be arranged to be removable, e.g., peeled away, such as after insertion of the medical device. For example, in an illustrative embodiment, after a patient has had ECMO support, an intravascular blood pump may be installed (e.g., via the first arm). After installing, the blood pump, the cannula may be removed from the patient, with just the blood pump remaining in the patient. In such embodiments, the access device may be attachable to a repositioning unit (no shown), which may be attachable to the patient at or near the insertion site.

According to another embodiment present disclosure, a kit may be provided. The kit may comprise or consist of any embodiment of an access device according to the first aspect of the present disclosure, an external medical device, such as an extracorporeal membrane oxygenation (ECMO) device configured to be coupled to the second arm of the single access device, and at least one medical device configured to be inserted through the first hemostatic valve, the first lumen, and the joint lumen of the access device. In some embodiments, the medical device may be an intravascular pump. The kit also may include a cannula attached to the access device. In some embodiments, the kit also may include a needle to enable the physician to gain access to the artery or vein. In some embodiments, the kit also may include a guidewire to enable placement of the cannula into the vasculature. The kit also may include one or more dilators at subsequent sizes to sequentially expand the vascular prior to insertion of the described device.

The basic components of such a kit can be seen in FIG. 7, where there is an access device 1, an external medical device 400 (e.g., an ECMO device), and at least one medical device 750 configured to be inserted through a part of the access device. In some embodiments, the kit may also include additional medical devices, such as one or more dilator assemblies, and/or one or more needles.

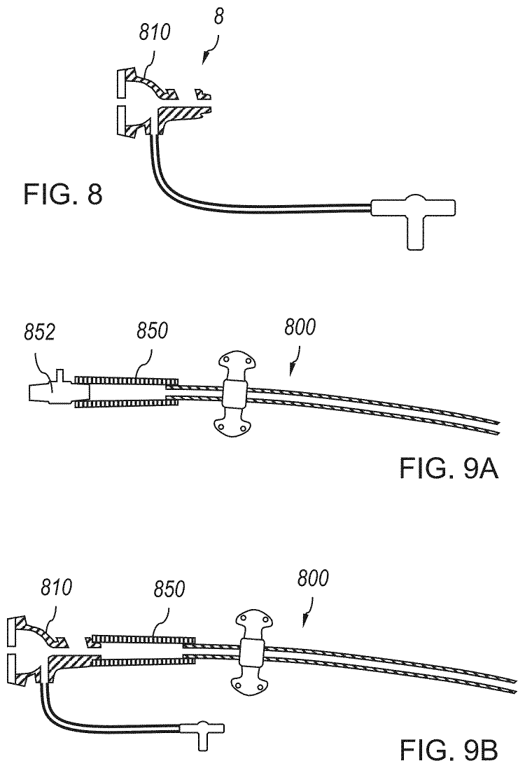

According to still another embodiment, the access device may be configured as a modular access system. For example, in such embodiments, the clinician may configure the access device according to the type (and order) of support needed by a patient. In a first embodiment, the access device 8 may be configured such that one or both arms are removably attachable to the hub. For example, as shown in FIG. 8 in some embodiments, the second arm (e.g., connected to the ECMO circuit) may be removable from the hub, such as after ECMO support has been completed. In such embodiments, the first arm 810 may remain attached to the hub while the patient is under VAD support. In a similar fashion, a clinician may begin using the hub with only the first arm attached when VAD support is needed, and then attach the second arm if/when ECMO support is then needed. In other embodiments, the clinician may attach just the second arm to the hub if only ECMO support is needed first, and thereafter attach the first arm to the hub if/when VAD support is needed. As will be appreciated in view of the above, the clinician may still decide to leave both the first and second arms attached to the hub, irrespective of which type of support is needed by the patient

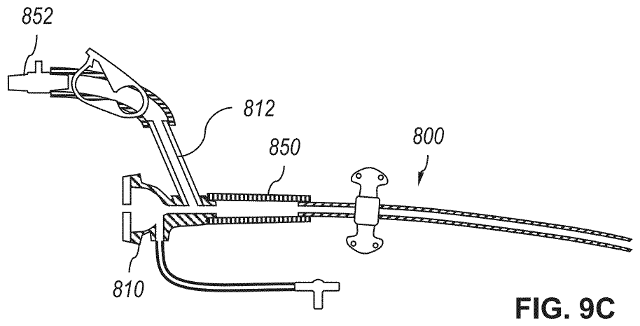

FIGS. 9A-9C illustrate embodiments in which a tubular extension 850 can be used to attach different configurations of the modular access device for patient support. For example, as shown in these views, the tubular extension 850 is attachable to the cannula 800, which can be inserted into the patient at the single insertion site (not shown). In embodiments in which only ECMO support is needed (or is needed first) the clinician may attach only a connector 852 to the tubular extension for ECMO support (see FIG. 9A). Once ECMO support has been completed, the connector can be removed, the clinician may attach the hub with only a single arm (e.g., the first arm 810) if/when VAD support is needed (see FIG. 9B). As will be appreciated, the clinician need not first attach the connector 852 for ECMO support to the tubular extension. Instead, if only VAD support is needed, the clinician can just the hub with the first arm to the tubular extension 850 (see FIG. 9B). Finally, as shown in FIG. 9C, the access device with both the first arm 810 and second arm 812 may be attachable to the tubular connector if simultaneous or tandem support is to be provided to a patient. As shown in this view, the connector 852 may be attached to the second arm 812 in some embodiments.

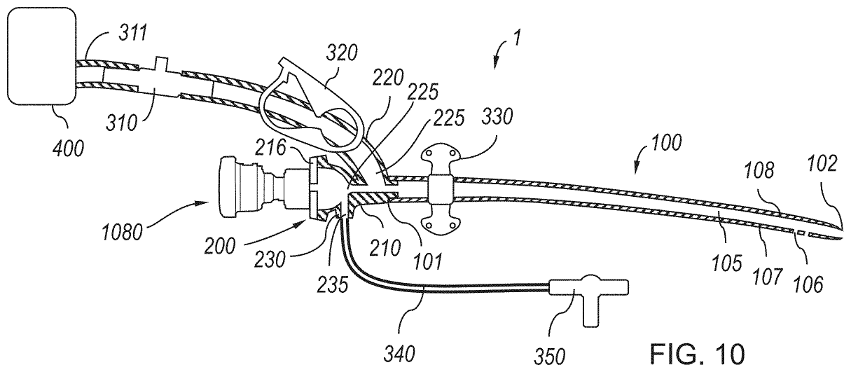

In still other embodiments, the device may include additional leak-protection features. For example, the hub may include an external leak-protection device (e.g., disposed on the outside of the hub) in some embodiments. In some embodiments, as shown in FIG. 10, the leak-protection feature may include a Tuohy Borst valve 1080. The leak-protection feature also may include an annular valve design with a mechanism that squeezes a seal around a catheter of a medical device (not shown) that may pass through annular valve. The leak-protection feature may further include, generally, a seal with a mechanism actuated either automatically or by the user to seals around such a medical device catheter to prevent a leak. As will be appreciated, other suitable valves and/or seals may be used to form the external leak-protection feature.

In some embodiments, the seal or valve may be configured to help immobilize the catheter of the medical device and/or other portion or accessory of the medical device (e.g., a repositioning sheath) from moving in and out of the hub and/or in and out of the shared cannula 100. The leak-protection feature also may include one or more locking features for locking a medical device and/or other portion or accessory of the medical device (e.g., a dilator) to the leak-protection feature. In some embodiments, the locking feature and/or the hub may include a feature for a sterile sleeve. In some embodiments, the medical device may include a catheter-based pump such as a heart pump, and the leak-protection feature design may include considerations for ease of use, ease of guidewire insertion, ease of pump insertion, ease of insertion for other accessories, ease of establishing the seal, leak protection effectiveness, and pump position retention effectiveness.

Although the hub shown in FIG. 10 includes only a single hemostatic valve, it will be appreciated that the leak-protection features may be added to a hub having two (or more) hemostatic valves. The leak protection features also may be added to an access device having a different configuration than that shown in FIG. 10.

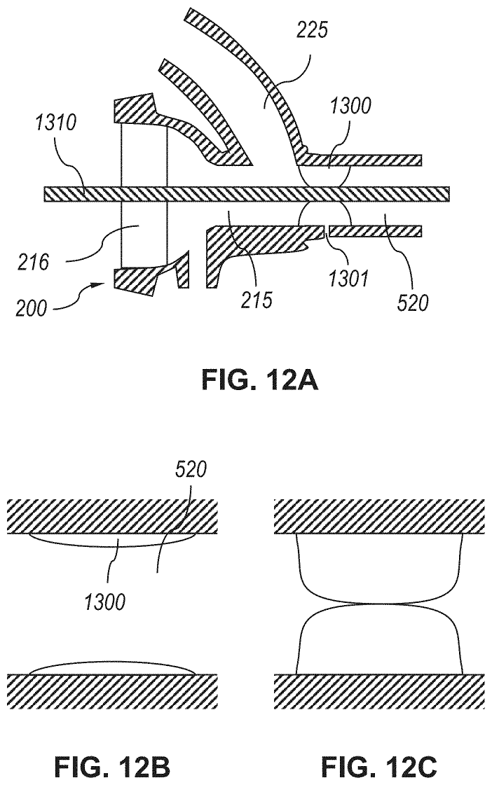

In some embodiments, the device may include a cannula with a bifurcation and a valve system. Referring to FIG. 12A, in some embodiments, the valve system may include an inflatable valve system. The valve system also may include a passive valve system. In some embodiments, the valve system may be configured to reduce flow disturbance. In FIG. 12A, the inflatable valve 1300 can be seen disposed within a lumen of the hub 200. As shown, the valve is disposed within the third lumen 520 (e.g., joint lumen), but as will be recognized, it may be placed in other locations within the hub as well, including the first lumen 215 or the second lumen 225. As shown, a medical device 1310 may be inserted through a hemostatic valve 216 and through the inflatable valve 1300. The valve may be inflated in any known manner. For example, in some embodiments, the valve may be inflated by providing a saline solution through a port 1301 coupled to an internal volume of space within the inflatable valve. The valve may have a plurality of configurations. For example, as seen in FIG. 12B, in a deflated configuration, the lumen in which the valve is placed may be substantially unobstructed. Referring to FIG. 12C, in an inflated configuration, the lumen in which the valve is placed may be completed blocked or may be configured to hold one or more medical device(s) in place and/or prevent flow past the valve except through whatever medical device(s) are in place. As will be appreciated, although the hub is shown as having a hemostatic valve and an inflatable valve, in other embodiments, the hub may include only an inflatable valve for blocking the third (e.g., joint) lumen and for allowing passage of the medical device into the patient (e.g., via the first and third lumens).

In other embodiments, the device may include an internal bladder. In such embodiments, the bladder may be configured to act like a flap that under forward blood flow may push to the side against a medical device, such as a mechanical circulatory device (e.g., a sheath or cannula), so that flow does not interfere with the medical device (e.g., if clotting or hemolysis is a concern).

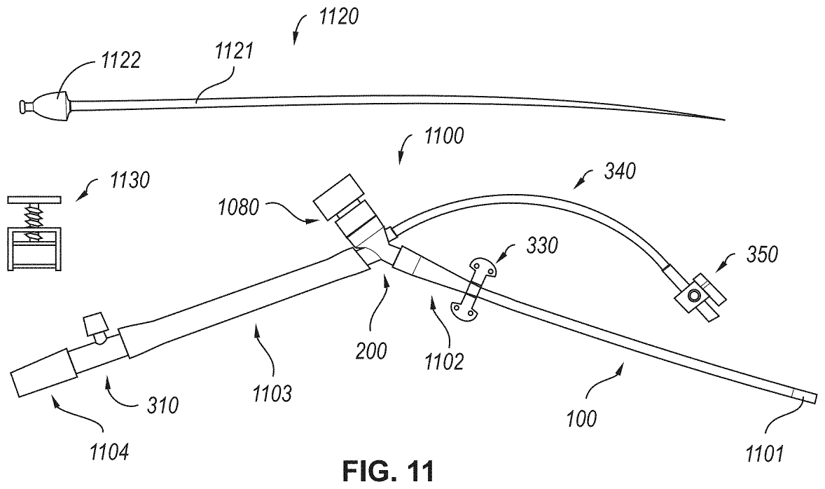

Referring to FIG. 11, an embodiment of another access system 1100 can be seen. As shown, hub 200 may be operably coupled to a cannula 100. The cannula may include a reinforcement cage 1101 at the distal end. The cannula also may include a coupling 1102 at the proximal end for connecting to the hub. A fixation feature 330 may be attached to the cannula, such as to attach the access system to a patient. Tubing 340 (e.g., a high flow side port) may be used to couple the hub and a valve 350. (e.g., via a third arm). Flexible tubing 1103 (e.g., perfusion tubing) may be removable coupled to the hub (e.g., via a second arm). A connector 310 (such as a ⅜″ barbed connector) may be coupled to a proximal end of tubing 1103. A tubing cap 1104 may be coupled to the connector. A clamp 1130 may be used, e.g., to control flow through flexible tubing 1103.

A dilator 1120, including a tubular member 1121 attached coupled to a dilator handle 1122, may be present. In some embodiments, as disclosed herein, the tubular member may pass through an arm (e.g., the first arm) of the hub 200 into the cannula, while the dilator handle remains proximal to the hub 200. For example, in some embodiments, the tubular member 1121 of the dilator may be passed through a Touhy Borst valve 1080 coupled to a proximal end of the hub 200, passing into the first arm and then into cannula. In some embodiments, the dilator may be used to facilitate insertion of a medical device into the patient (e.g., via the first arm)

Those skilled in the art will recognize or be able to ascertain using no more than routine experimentation many equivalents to the specific embodiments of the invention described herein. Such equivalents are intended to be encompassed by the following claims.

Embodiments of the present disclosure are described in detail with reference to the figures wherein like reference numerals identify similar or identical elements. It is to be understood that the disclosed embodiments are merely examples of the disclosure, which may be embodied in various forms. Well known functions or constructions are not described in detail to avoid obscuring the present disclosure in unnecessary detail. Therefore, specific structural and functional details disclosed herein are not to be interpreted as limiting, but merely as a basis for the claims and as a representative basis for teaching one skilled in the art to variously employ the present disclosure in virtually any appropriately detailed structure.

1. An access device, comprising:
a hub configured to be coupled to a proximal end of a cannula having a proximal end, a distal end and a joint lumen therethrough, the hub comprising:
a first arm having a first lumen and a first hemostatic valve, the first lumen operably connected to the joint lumen, the first lumen and the first hemostatic valve configured for passage of an internally placeable medical device;
a second arm coupled to the first arm, the second arm having a second lumen operably connected to the joint lumen, the second arm configured to be operably coupled to an external medical device.

2. The access device according to claim 1, wherein the external medical device includes an extracorporeal membrane oxygenation (ECMO) device.

3. The access device according to claim 1, wherein the cannula is coupled to the hub via a threaded or a barbed connection.

4. The access device according to claim 1, wherein the cannula is a joint cannula, the joint cannula comprising:
a first joint cannula section having a proximal end and a distal end, the proximal end of the first joint cannula section being coupled to the hub; and
a second joint cannula section having a proximal end and a distal end, the proximal end of the second joint cannula section being coupled to the distal end of the first joint cannula section.

5. (canceled)

6. The access device according to claim 1, wherein the second arm is configured to be operably coupled to an external medical device via an additional cannula.

7. (canceled)

8. The access device according to claim 1, wherein the joint lumen has an inner diameter ID, wherein 3 mm≤ID≤36 mm.

9-10. (canceled)

11. The access device according to claim 1, wherein a first angle formed between a central axis of the first arm and a central axis of the second arm is less than 90 degrees.

12. The access device according to claim 1, wherein a second angle formed between a central axis of the first lumen and a central axis of the second lumen is less than 90 degrees.

13. The access device according to claim 11, wherein the first angle, the second angle, or both are between 15 degrees and 60 degrees.

14. The access device according to claim 1, wherein the first lumen and second lumen connect to form a junction.

15. The access device according to claim 1, further comprising a clamp configured to allow a user to clamp off the second arm.

16. The access device according to claim 1, further comprising a fixation feature.

17-20. (canceled)

21. The access device according to claim 1, wherein the cannula is reinforced with coiled wire, braided wire, or a precision-cut hypotube.

22-26. (canceled)

27. The access device according to claim 1, wherein the cannula further comprises at least one lumen in a distal portion of the cannula, the at least one lumen extending from an outer surface of the cannula to the joint lumen through a wall of the cannula.

28. The access device according to claim 1, wherein the cannula is configured to receive a dilator assembly.

29. The access device according to claim 1, wherein the hub further comprises a third lumen operably connected to the first lumen, the second lumen, or both.

30-33. (canceled)

34. The access device according to claim 1, wherein the medical device is a guide wire, a balloon catheter, or a catheter-based heart pump.

35. The access device according to claim 1, further comprising a second hemostatic vale positioned in the first arm.

36. The access device according to claim 1, further comprising an inflatable valve disposed within the first lumen, second lumen, or joint lumen.

37. A method for using a single access device, comprising:
providing an access device according to claim 1;
connecting the hub of the access device to the cannula; and
inserting a medical device into the first arm of the single access device and into a patient;
coupling an external medical device to the single access device via an additional cannula.

38-40. (canceled)

41. A kit comprising:
an access device according to claim 1;
an external medical device configured to be coupled to the second arm of the access device; and
a medical device configured to be inserted through the first hemostatic valve, the first lumen, and the joint lumen.

42-47. (canceled)
